(19) United States
(12) Patent Application Publication
(10) Pub. No.: US 2023/0226258 A1
(43) Pub. Date: Jul. 20, 2023

(54) Wearable Engineered Human Skin and Systems and Methods for Making the Same

(71) Applicant: The Trustees of Columbia University in the City of New York, New York, NY (US)

(72) Inventor: Hasan Erbil ABACI, New York, NY (US)

(73) Assignee: The Trustees of Columbia University in the City of New York, New York, NY (US)

(21) Appl. No.: 18/115,402

(22) Filed: Feb. 28, 2023

Related U.S. Application Data
(63) Continuation of application No. PCT/US2021/049671, filed on Sep. 9, 2021.
(60) Provisional application No. 63/077,029, filed on Sep. 11, 2020.

(51) Int. Cl. A61L 27/38; C12N 5/071

(52) U.S. Cl. CPC A61L 27/3813; A61L 27/3891; C12N 5/0625; C12N 2501/50

(57) ABSTRACT

Engineered skin substitutes comprising an outer-facing portion and an inner-facing portion and methods of making the same are provided. The skin substitutes are configured to conform to a shape and a dimension of a body part of a subject, and have at least one surface that circles back on itself so as to enclose at least a portion of the body part. In some instances, dermis and epidermal layers can be formed in an air liquid interface. The exemplary skin substitutes are wearable and can be made to conform to a generic body part or a specific body part from a three-dimensional representation of the body part.

### CROSS REFERENCE TO RELATED APPLICATIONS

This Application is a continuation of International Application PCT/US2021/049671, filed Sep. 9, 2021, which claims priority to and the benefit of U.S. Provisional Application 63/077,029, filed Sep. 11, 2020, each of which is incorporated herein by reference in its entirety.

### STATEMENT REGARDING FEDERALLY SPONSORED RESEARCH OR DEVELOPMENT

This invention was made with government support under 5K01AR072131 awarded by the National Institutes of Health (NIH). The government has certain rights in the invention.

### BACKGROUND

Each year, more than one million patients are hospitalized in the U.S. for significant skin loss due to thermal and pressure injuries, chronic diabetic ulcers, or genetic skin diseases. The ability to generate engineered human skin substitutes (HSSs) is a potential therapy for these patients (Abaci et al, 2017, Exp Biol Med).

Patients with significant skin loss due to traumatic injury, burns or genetic diseases (e.g., epidermolysis bullosa) are currently treated by grafting engineered skin substitutes that come as rectangular or circular planar patches that have to be stitched together to cover the wound area (Boyce ST et al. Ann Surg. 2002; Hirsch T et al. Nature. 2017). However, when the area is large, or has an irregular shape and/or curvature, the engraftment of conventional skin substitutes becomes laborious, requiring long procedure times due to extensive graft placement and suturing during surgery, and typically leads to ineffective coverage of the wound area.

Existing planar HSSs are typically grafted as multiple patches on different parts of the body, including irregular parts like fingers or facial features, requiring a high number of sutures in between the patches or extensive bandaging to cover the entire wound area. Using multiple HSS patches on a curved or an irregularly-shaped body part has significant disadvantages. For example, the HSS patches may not fully integrate with each other, and one or more patches can fail over the long term. In addition, the appearance of multiple patches connected with a high number of sutures may not be desirable. Use of multiple HSS patches may not provide natural mobility for the body part and the patient may have restrictions regarding activities such as tennis, swimming or running. The patient may be unable to complete simple tasks, such as holding objects and walking, due to extensive suturing used to cover up the wound with multiple patches and the likelihood of sutures to tear through, leading to an open wound.

### SUMMARY

The problems noted above can be addressed using skin substitutes configured to conform to the shape of a body part or the shape of a body part of a particular patient as, for example, a single piece. The problems noted above can also be addressed using 3D human skin substitutes in personalized 3D shapes that allow a patient to seamlessly wear or place the skin substitute on a target location (e.g., on a wound).

Aspects described herein provide a skin substitute having an outer-facing portion and an inner-facing portion, wherein the skin substitute is configured to conform to a shape and a dimension of a body part of a subject, and wherein the skin substitute has at least one surface that circles back on itself so as to enclose at least a portion of the body part.

Further aspects provide a first method of making a skin substitute by forming the skin substitute on or in a hollow and porous scaffold. The skin substitute is configured to conform to a shape and a dimension of a body part of a subject and have at least one surface that circles back on itself so as to enclose at least a portion of the body part.

Aspects described herein provide a second method of forming a wearable skin substitute by (1) obtaining a three-dimensional model of a target region of a subject’s body, (2) forming, based on the three-dimensional model, a hollow, porous, and perfusable scaffold that conforms to the target region, the scaffold having an outer surface and a plurality of pores, (3) forming, based on the three-dimensional model, a chamber having an inner surface dimensioned to enclose the outer surface of the scaffold, with a spacing of 2 to 7 mm between the outer surface of the scaffold and the inner surface of the chamber, (4) positioning the scaffold inside the chamber, (5) forming a dermis in the chamber by introducing a dermis solution comprising a collagen gel and dermal fibroblasts into the chamber, wherein the dermis is formed around the scaffold, (6) seeding epidermal cells on the dermis in the chamber, and (7) perfusing the scaffold with medium to form an air-liquid interface culture.

### BRIEF DESCRIPTION OF DRAWINGS

FIG. 1 shows an exemplary conventional method of producing conventional human skin substitutes (HSS) that have open edges;

FIG. 2A shows exemplary skin scaffolds in a cylindrical shape, a mouse hindlimb shape, and a human hand shape;

FIG. 2B shows exemplary design parameters for an exemplary cylindrical skin scaffold having an inlet and outlet port and pores;

FIG. 3 shows an exemplary skin chamber with inlet and outlet ports around an exemplary skin scaffold with inlet and outlet ports and an exemplary step of making the dermis in the skin chamber around the skin scaffold;

FIG. 4 shows an exemplary step of seeding epidermal cells on the dermis in the skin chamber followed by rotating the skin chamber;

FIG. 5A shows an exemplary apparatus for perfusion and vascularization of the wearable engineered skin;

FIG. 5B provides an exemplary graph of the glucose concentration at the fingertips as a function of medium perfusion rate of an exemplary skin glove during the course of epidermilization;

FIG. 5C provides an exemplary representation of glucose concentration along the fingertips of the exemplary skin scaffold having a suspended hand-shaped skin scaffold during the course of epidermilization;

FIG. 5D provides an exemplary stained dermis and epidermis layer formed on the skin scaffold of FIG. 5C (scale bars: 25 µm);

FIGS. 6A-6C show exemplary incision and suture sites for skin substitutes shaped like a cylinder, a hand, and a mouse hind leg, respectively;

FIGS. 6D-6E show cross sections of formed skin substitutes stained to show the presence of epidermis proteins (scale bars: 50 µm);

FIG. 7A shows exemplary cross sections of Wearable Engineered Human Skin (WEHS) in accordance with aspects described herein stained for dermal ECM (extracellular matrix) proteins and compared to conventional skin substitutes stained for dermal ECM proteins (scale bars: 50 µm);

FIG. 7B shows exemplary cross sections of WEHS in accordance with aspects described herein stained to show the presence of epidermal basement membrane proteins compared to conventional skin substitutes stained to show the presence of epidermal basement membrane proteins (scale bars: 50 µm);

FIG. 7C shows the mean fluorescence intensity of proteins related to dermal ECM from FIG. 7A in WEHS (dark gray bars) compared to conventional skin substitutes (light gray bars);

FIG. 7D shows the mean fluorescence intensity of proteins related to epidermal basement membrane proteins from FIG. 7B in WEHS (dark gray bars) compared to conventional skin substitutes (light gray bars);

FIGS. 8A-8B show exemplary results of mechanical stress tests of conventional (FIG. 8A) and WEHS in accordance with aspects described herein (FIG. 8B);

FIGS. 8C-8D show exemplary results of rupture stress tests of conventional (FIG. 8C) and WEHS in accordance with aspects described herein (FIG. 8D); and

FIG. 8E shows exemplary Young’s Modulus measurements for conventional skin substitutes compared to WEHS.

### DETAILED DESCRIPTION

All references cited herein, including but not limited to patents and patent applications, are incorporated by reference in their entirety.

Conventional skin substitutes are planar and are not designed or made to conform to a subject’s body parts. As a result, grafting conventional skin substitutes is time consuming and difficult. Conventional skin substitutes are formed from engineered epidermis that must be adapted to conform to flat and curved body parts and therefore do not feel or function like normal skin.

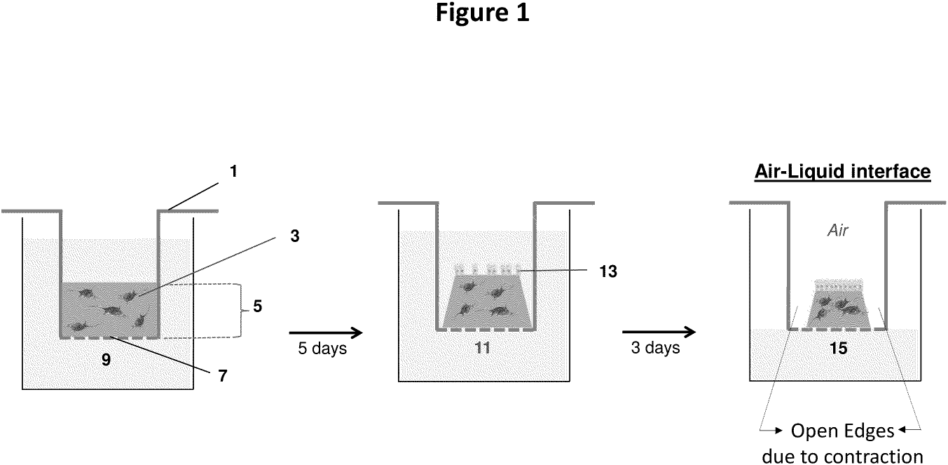

FIG. 1 depicts a conventional approach for forming skin substitutes in a transwell 1 by encapsulating fibroblasts 3 into a 3D hydrogel (e.g., collagen type I) 5 over a porous membrane 7 suspended in fibroblast medium 9. After 5 days, the skin substitute is placed in epidermilization medium 11 and seeded with keratinocytes 13. After three days the skin substitute is transferred to cornification medium 15 and forms an air-liquid interface. The resulting planar skin substitute has open edges due to contraction. These conventional skin substitutes need to graft on to the shape of a body part in need of a substitute rather than being configured to conform to a specific target location (e.g., body part). Conventional skin substitutes can be made according to exemplary methods known in the art. See, e.g., Abaci, H. E. et al. Human Skin Constructs with Spatially Controlled Vasculature Using Primary and iPSC-Derived Endothelial Cells. Adv. Healthc. Mater. 5, 1800-1807; P. Gangatirkar, S. Paquet-Fifield, A. Li, R. Rossi, P. Kaur, Nat. Protoc. 2007, 2, 178.

To address the need for skin substitutes that conform to the shape of a body part, 3D WEHS in custom shapes are provided that can be directly worn on any part of the body with a regular shape (e.g., arms) or irregular shape (e.g., hand, face) with curved features.

In some instances, the WEHS described herein are generated in an enclosed geometry in order to mimic the physiological mechanical forces in skin development. In contrast, conventional HSSs have open edges. Therefore, the skin substitutes described herein provide dermis and epidermis layers having functionality closer to real skin than those generated by conventional methods.

Aspects described herein can meet the medical needs of patients requiring skin transplants on their hands, feet, joints (e.g., elbows, knees) and face, by wearing or placing the engineered skin constructs on a target location (e.g., skin gloves on a hand, a wound).

Significant skin loss can occur due to a variety of causes (e.g., thermal and trauma-related injuries, chronic diabetic ulcers or genetic skin diseases, such as epidermolysis bullosa (EB)). Recessive dystrophic EB (RDEB) is a severe type of EB, in which reduced collagen VII accompanied by recurrent blistering and scarring of the hands and feet leads to fusion of fingers and toes and a mitten-like deformity where the hand becomes encased in an epidermal cocoon in early childhood. Engineered HSSs made from in vitro-expanded donor cells, patients’ revertant cells, and gene-corrected iPSC-derived cells offer some clinical promise to treat these patients. However, regardless of the cell source, the current technology creates HSSs as rectangular or circular flat patches that have to be cut individually and wrapped around each finger, and stitched together to sufficiently cover a target location. This process significantly lengthens the surgery time and worsens the aesthetic and functional outcome of the procedure.

Aspects described herein address this long-lasting handicap of conventional HSSs by reimagining the engineered skin substitutes as wearable 3D enclosed tissues, and by developing a 3D-printing approach to generate WEHS in custom shapes to fit irregularly shaped parts of the body as a single-piece (e.g., skin gloves). For example, WEHS can be designed specifically for an RDEB patient’s hand and surgically delivered as a biological glove only requiring sutures around the wrist area to close, for example, a wound, as opposed to conventional HSSs which require greater numbers of sutures between individual rectangular patches.

WEHS in customizable enclosed 3D shapes as described herein can effectively cover and treat wounds on target locations on the body that are irregularly shaped. In addition, since WEHS have an enclosed geometry (as opposed to conventional HSSs with open edges), it is believed that WEHS better mimic the physiological biomechanical forces in skin morphogenesis, and thereby generate a more robust dermis and epidermis than those generated by the conventional method.

As described herein the capabilities of 3D-printing technology can be applied to create a custom-shaped, hollow (e.g., perfusable) and porous (e.g., permeable) skin scaffold that allows for the generation of WEHS in an enclosed and defined 3D geometry.

In some instances, pre-vascularizing WEHS with skin-specific endothelial cells can be used to transplant vascularized WEHS onto the hindlimbs of rats or larger animal models as a wearable graft (e.g., skin sleeves). In some aspects, an automated and systematic workflow for the generation WEHS for different parts of the body in human-scale, such as hands, feet and face can be used.

In further aspects, WEHS can be made in various geometries and scales using primary human cells. The functioning of the WEHS as skin grafts in rats and larger animal models can be used to further validate methodologies. WEHS using gene-corrected induced pluripotent stem cell (iPSC)-derived skin cells of RDEB patients or revertant cells from mosaic RDEB patients can be developed and used in larger animal models. WEHS technology as described herein can transform the medical management of EB patients and patients with skin injuries, and significantly improve the lives of people awaiting skin transplants.

Previous skin graft technology is based on layering or embedding skin cells on or in a planar substrate (e.g., Collagen gel) and letting the skin cells contract freely during remodeling due to non-constrained edges, resulting in a planar tissue with non-physiological dermal contraction. Aspects described herein provide 3D WEHS with a defined customizable shape that has an enclosed or semi-enclosed geometry (mimicking the fully enclosed human skin) allowing for physiologically-relevant biomechanical forces to be applied to the dermis and for transplantation of the WEHS as a wearable skin substitute (e.g., skin gloves, skin vest etc.). This capability of WEHS significantly reduces the number of sutures and time required for the placement of skin grafts onto a target location during surgery.

Aspects described herein differ from pre-existing perfusable and custom-shaped tissue models in permitting perfusion and diffusion of nutrients through the surface pores with a density, diameter, thickness and geometry optimized for skin generation and maintenance, and for determining the final desired shape of the engineered skin tissue.

In contrast, prior methods rely on seeding a monolayer of endothelial cells or embedding endothelial cells in the dermal compartment of HSSs. The previous methods do not permit direct perfusion over these cells after their incorporation into HSSs and therefore do not mimic physiological blood flow over endothelial cells. In contrast, vascularization methods described herein can optionally perfuse skin endothelium prior to their engraftment on to a body part of a patient.

Aspects described herein provide 3D human skin substitutes in personalized and enclosed 3D shapes that allow the patients to simply wear or place them on a target location on a body part including but not limited to the face, nose, ears, hands, fingers, feet, toes, elbows, knees or chest. In addition, this capability of WEHS significantly reduces the number of sutures and time required for the placement of skin grafts onto a target location during surgery.

The WEHS described herein provide improved and more robust skin substitutes with a better dermis and epidermis, compared to conventional HSSs, by recreating the physiologically relevant biomechanical forces in the skin development due to the fully enclosed geometries enabled by the method.

In addition, the WEHS described herein can have a perfusable vasculature that can promote graft viability and integration.

Further uses of the WEHS described herein include in vitro modelling of skin diseases and as drug screening platforms. Since the methods described herein provide continuous medium flow and a dermis coated with endothelial cells, the technology can be used to evaluate systemic or topical delivery of drugs to or from the skin by injecting the drug into the medium or applying it topically, respectively.

Aspects described herein provide a skin substitute having an outer-facing portion and an inner-facing portion, wherein the skin substitute is configured to conform to a shape and a dimension of a body part of a subject, and wherein the skin substitute has at least one surface that circles back on itself so as to enclose at least a portion of the body part.

The term “skin substitute” refers to a replacement or augmentation for human or animal skin tissue formed from natural (e.g., human or animal cells, support tissue) or artificial (e.g., biocompatible plastic or other compounds) components or a combination of natural and artificial components configured to replace or augment human or animal skin in situ.

The term “outer-facing portion” refers to a portion of a skin substitute that is oriented away from the body (e.g., toward the air). The term “inner-facing portion” refers to a portion of a skin substitute that oriented toward the body tissues. The inner-facing portion can be oriented to be opposite the outer-facing portion.

Some embodiments described herein have at least one surface that circles back on itself so as to enclose or partially enclose a body part. For example, in the context of a skin substitute shaped like a glove, the surface at each knuckle circles back on itself so as to enclose a portion of the respective finger, and the surface at the center of the palm circles back on itself so as to enclose the palm.

The term “conform to a shape and a dimension of a body part” refers to a skin substitute that is configured to fit or substantially fit (e.g., 50, 60, 70, 80, 90% fit) over an entire surface or a portion of a surface or dimension of a body part. In contrast, previous methods require stitching together two or more conventional and generic planar HSSs in order to conform to and cover an entire body part.

In some instances, the outer-facing portion is an epidermal portion, and the inner-facing portion is a dermal portion. The epidermal portion can comprise epidermal cells (e.g., keratinocytes, melanocytes, Langerhan cells, and epidermal stem cells).

In some instances, the dermal portion comprises dermal cells (e.g., fibroblasts, mesenchymal cells, dermal papilla cells, adipocytes, sensory neurons, mesenchymal stem cells, endothelial cells, smooth muscle cells, and pericytes).

In some instances, the skin substitute is formed on a hollow and porous scaffold, and the scaffold is printed with a 3D Printer. The scaffold can be made of a material selected from one or more of 3D-printable thermoplastic materials selected from the group consisting of acrylonitrile butadiene styrene, polycarbonate, glass, ceramic, polyamide, poly-lactic acid, epoxy resins, ceramic and alloys thereof, and 3D-printable photopolymers (e.g., Nylon 12, MED610 (Stratys), and KeySplint Soft (keyprint)).

The scaffold can have a plurality of pores, and the pores can have an average diameter of 5 to 500 µm to permit perfusion of the scaffold.

The body part to be covered or substantially covered by the skin substitute can be selected from the group consisting of a hand, one or more fingers, a foot, one or more toes, a face or a portion of a face, a head or a portion of a head, an ear or a portion of an ear, a limb or a portion of a limb, and a joint or a portion of a joint.

In some instances, the skin substitute can be explanted from the scaffold and transplanted on to the body part or a portion of the body part.

Further aspects provide a first method of making a skin substitute by forming the skin substitute on or in a hollow and porous scaffold. The skin substitute has an outer-facing portion and an inner-facing portion, and has at least one surface that circles back on itself so as to enclose or partially enclose a body part.

In some instances of the first method, a three-dimensional data representation of the body part is obtained (e.g., by laser scan of the body part). The scaffold can be formed from the three-dimensional data representation of the body part.

In some instances of the first method, the body part is selected from the group consisting of a hand, one or more fingers, a foot, one or more toes, a face or a portion of a face, a head or a portion of a head, an ear or a portion of an ear, a nose or a portion of a nose, a limb or a portion of a limb, a scalp or a portion of a scalp, and a joint or a portion of a joint.

In some instances of the first method, the scaffold further comprises an inlet port and an outlet port arranged so that a liquid can be introduced into and removed from an interior of the scaffold, wherein the liquid forms an air/liquid interface at one or more walls of the scaffold. The liquid can include cells, cell culture medium, and other supplemental components as desired to form the desired tissue.

In some instances of the first method, the liquid comprises one or more of dermis culture medium, epidermis culture medium, cornification medium, endothelial cell culture medium, and skin and vasculature co-culture medium.

In some instances of the first method, a chamber for receiving the scaffold is formed or used, and the skin scaffold can be placed into the chamber. In some instances, a hydrogel containing dermal cells can be introduced into the chamber. A dermal layer can then be formed on the scaffold. In some instances of the first method, the dermal layer is formed for about 1 to about 2 weeks in a dermis culture medium.

The dermal cells can be selected from one or more of fibroblasts, mesenchymal cells, dermal papilla cells, adipocytes, sensory neurons, mesenchymal stem cells, endothelial cells, smooth muscle cells, pericytes.

In some instances of the first method, epidermal cells are introduced into the chamber, and an epidermal monolayer is formed on the scaffold in epidermis culture medium. In another aspect, laminin and fibronectin can be introduced into the chamber.

In some instances of the first method, the chamber is rotated for about 4 to about 5 hours after introducing the epidermal cells into the scaffold. The epidermal cells can be selected from the group consisting of keratinocytes, melanocytes, Langerhan cells, and epidermal stem cells.

In some instances of the first method, an interior of the scaffold is perfused with cornification medium, and the epidermal monolayer is formed at the air-liquid interface.

Aspects described herein provide a second method of forming a wearable engineered human skin by (1) obtaining a three-dimensional model of a target region of a subject’s body, (2) forming, based on the three-dimensional model, a hollow, porous, and perfusable scaffold that conforms to the target region, the scaffold having an outer surface and a plurality of pores, (3) forming, based on the three-dimensional model, a chamber having an inner surface dimensioned to enclose the outer surface of the scaffold, with a spacing of 2 to 7 mm between the outer surface of the scaffold and the inner surface of the chamber, (4) positioning the scaffold inside the chamber, (5) forming a dermis in the chamber by introducing a dermis solution comprising a collagen gel and dermal fibroblasts into the chamber, wherein the dermis is formed around the scaffold, (6) seeding epidermal cells on the dermis in the chamber, and (7) perfusing the scaffold with medium to form an air-liquid interface culture.

The term “three-dimensional model” refers to a mathematical representation of the three-dimensional surfaces of an object.

The term “dermis solution” refers to media or cell-culture medium for promoting the growth of dermis and cell types that make up dermis. Dermis solution can contain nutrients, growth factors and other components used by keratinocytes, endothelial cells, or other cell types to proliferate and grow in a chamber, on a scaffold, or another structure. Commercially available dermis solutions can include, for example, collagen type I, gelatin, collagen type IV, fibronectin, hyaluronic acid, laminin, fibrinogen, Matrigel, alginate, chitosan, silk, or decellularized human skin ECM, or combinations of these as the main hydrogel in the dermis solution. Dermis solution can also include cell culture medium (e.g., DMEM/F12, pH modifiers (e.g., NaOH), fetal bovine serum (FBS), and dermal cells (e.g., dermal fibroblasts). See, e.g., P. Gangatirkar, S. Paquet-Fifield, A. Li, R. Rossi, P. Kaur, Nat. Protoc. 2007, 2, 178.

In some instances of the second method, a density, a size, and a distribution of the pores on the surface of the scaffold are configured to permit diffusion of cell culture medium inside the scaffold to the dermis exposed to air.

In some instances, the second method further comprises perfusing the dermis with endothelial medium comprising endothelial cells and a growth factor.

In some instances of the second method, a density of the endothelial cells in the endothelial medium is at least 5 million cells/ml.

In some instances of the second method, the endothelial cells in the endothelial medium are incubated with the dermis for at least 3 hours at 37° C. in a static culture and attached to an inner wall of the dermis through the pores.

In some instances of the second method, the endothelial cells in the endothelial medium are stimulated to form spontaneous vessel-like structures from the pores by the growth factor for at least two days prior to grafting the wearable skin substitute to the target region.

In some instances of the second method, the step of forming a scaffold comprises 3D printing the scaffold, and the step of forming the chamber comprises 3D printing the chamber. In some instances, the material used for the printed three-dimensional object is biocompatible material suitable for use in treating a subject.

In some instances of the second method, the inner surface of the chamber is dimensioned so that the spacing between the outer surface of the scaffold and the inner surface of the chamber is 3 to 5 mm and is substantially uniform. The term “substantially uniform” as used herein in reference to the spacing between the outer surface of the scaffold and the inner surface of the chamber refers to spacing between the outer surface of the scaffold and the inner surface of the chamber that vary by less than 20%, 10%, or 5%.

In some instances of the second method, the scaffold comprises an inlet port and an outlet port for perfusing the scaffold with medium. For example, dermis solution, endothelial medium, or other solutions can be provided to the scaffold through the inlet port and removed from the scaffold through the outlet port.

In some instances of the second method, the dermis solution comprises neutralized collagen type I gel and dermal fibroblasts. Alternatively, the main hydrogel of dermis solution can comprise collagen type I, gelatin fibrinogen, Matrigel, alginate, chitosan, silk, or decellularized human skin ECM, or combinations of these. Additional components that can be included in the dermis solution may include elastin, collagen type IV, fibronectin, hyaluronic acid, and laminin. In some instances of the second method, the concentration of neutralized collagen type I gel in the dermis solution is 3 mg/ml.

In some instances of the second method, a final cell density of dermal fibroblasts in the dermis solution is 250,000 cells/ml. In some instances, the dermis solution is introduced into the chamber, the scaffold is incubated in the dermis solution, and the dermis solution forms a gel around the scaffold. In some instances, the scaffold is incubated in the dermis solution for about an hour at 37° C., forming a dermis.

In some instances of the second method, the formed dermis is removed from the chamber and incubated in a fibroblast culture medium. In some instances, the formed dermis is incubated in the fibroblast culture medium for at least two weeks. Alternatively, the formed dermis can be incubated for a shorter period of time (e.g., at least 2, 3, 4, 5, 6, 7, 8, 9, 10 or more days) or a longer period of time (e.g., 3, 4, 5 weeks or longer).

In some instances of the second method, the formed dermis is placed into the chamber and keratinocyte culture medium comprising keratinocytes are added to the chamber. In some instances, the keratinocyte culture medium comprises 3 to 5 million keratinocytes.

In some instances of the second method, the chamber is rotated continuously on a rotating platform for at least 4 hours at 37° C. Alternatively, the chamber can be rotated discontinuously with breaks between period of continuous rotation. In some instances, the chamber can be rotated at any suitable speed (e.g., 1-10 rotations/minute, 5 rotations/min).

### EXAMPLES

### Example 1 - Exemplary Steps to Form Wearable Engineered Human Skin

The steps below describe an exemplary method to form wearable engineered human skin (WEHS) configured to be used on a predetermined target location on a subject (e.g., burn or wound). The WEHS do not require stitching together pre-formed conventional planar or curved skin substitutes, form more natural dermis, and are able to withstand mechanical and rupture stress better than conventional skin substitutes.

Step 1: 3D scan a target location (e.g., hands) and create identical 3D “skin scaffold” models. As a proof-of-concept, a cylindrical shape was used. In this example, the skin scaffold is hollow, porous, and perfusable in order to form the skin substitute in an air-liquid-interface exposed to culture medium on the dermal side below, and to air from the epidermal side above. The air-liquid interface can permit formation of the epidermis layer.

In this example, the skin scaffolds have one inlet and one outlet port to allow for perfusion with cell culture media inside. The density, size and uniform distribution of the pores on the surface of the skin scaffold can be adjusted to permit a sufficient amount of diffusion of cell culture medium inside the scaffold, for example, to the epidermis exposed to the air.

Step 2: A 3D-printer (Stratys; Material:VeroWhite) was used to create skin scaffolds, and the supporting material was dissolved in 5 mM sodium hydroxide solution in water to create the hollow shape and the pores on the surface.

The skin scaffold was coated with 5% gelatin at 4° C. overnight and crosslinked it with transglutamase at 37° C. for 2 hours. The gelatin coating prevents undesired leakage of culture medium through the pores at later steps in the process when perfusion starts.

Step 3: A skin chamber was made of PDMS (polydimethylsiloxane) in the same shape of the scaffold as a receptacle to form the dermis around the scaffold. The skin chamber is slightly larger than and encases skin scaffold (e.g., with a spacing of 2-7 mm between the outer surface of the scaffold and the inner surface of the chamber). In some preferred embodiments, the spacing between the outer surface of the scaffold and the inner surface of the chamber is uniform (e.g., at a distance of 4 mm, or between 3-5 mm).

The skin scaffold was introduced into the skin chamber in a suspension containing a mixture of collagen type I gel and dermal fibroblasts and incubated for 1 hour to gel.

The formed dermis and skin scaffold was taken off the skin chamber, transferred to a standard tissue culture dish (e.g., petri-dish), and submerged in a fibroblast culture medium for 2 weeks for the formation and remodeling of the dermis (submerged culture period). During the submerged culture period, dermal fibroblasts reorganized the collagen fibers, produced new dermal ECM proteins (e.g., fibronectin and laminin), and contracted the dermis in the direction perpendicular to the outer surface of the dermis, reducing the thickness of the dermis by 3-5 times of its original thickness (e.g., 0.2-3 mm).

Step 4: After 2 weeks, the skin scaffold with the formed dermis was placed back into the skin chamber to seed human neonatal keratinocytes on top. In this example, 3 million keratinocytes in keratinocyte culture medium were introduced into the skin chamber, and the skin chamber was rotated continuously for 4 hours at 37° C. for uniform seeding of keratinocytes on the surface of the dermis.

Step 5: After keratinocyte attachment, the assembly of skin scaffold, dermis and keratinocytes was submerged in epidermis culture medium by introducing the culture medium into the skin chamber for the proliferation of keratinocytes on the surface for 7 days.

To bring the whole skin substitute into an air liquid interface, the inlet and outlet ports of the skin scaffold were connected to plastic tubing, and the tissue was suspended in a glass bottle. The tubes were connected to a medium reservoir and the skin scaffold was perfused with cornification medium using a peristaltic pump. This step allows for the medium to perfuse the dermis inside and air exposure outside for differentiation of keratinocytes and formation of the epidermis.

After 10 days in the air-liquid interface (ALI), WEHS with proper dermis and epidermis were formed and were ready to use.

Vascularization of WEHS was performed to promote the integration and viability of the grafts. Endothelial cells were introduced through perfusion into the skin scaffold. Endothelial cells then attach on the inner walls of the dermis through the pores, and are stimulated by growth factors in the medium to form spontaneous vessel-like structures for 2-3 days prior to grafting.

Step 6: To explant the WEHS from the skin scaffold, a minimal surgical incision was made on the top surface near the inlet and outlet ports, and WEHSs are peeled off of the skin scaffold as an intact shape. The incision site depends on the shape of the target area. For example, for skin transplantation on the hands, a circular incision can be made only around the wrist area to take off the WEHS to be grafted onto a target site on a patient.

To use WEHSs for in vitro drug testing purposes instead of grafting, WEHSs can be kept on skin scaffolds under perfusion. Chemicals or drugs can be directly added to the medium or on the epidermis to mimic systemic or topical treatment, respectively.

### Example 2 - Mouse Hindlimb Model

In another example, a WEHS can be formed for use on a mouse hindlimb. A 3D-laser scanned image of mouse hindlimbs was used to create an analogous 3D CAD model and converted to a hollow and porous shape. The 3D CAD model was 3D-printed using a polycarbonate-like material (MED610; Stratys) to serve as the skin scaffold. The skin scaffold was suspended at the center of a skin chamber and made of PDMS (polydimethylsiloxane).

The dermis was formed by pipetting a solution of collagen type I gel with 250,000 human dermal fibroblasts/ml into the skin chamber and allowing the solution to solidify around the skin scaffold at 37° C. The skin scaffold was submerged in fibroblast culture medium for 1-2 weeks. The dermis formed around the skin scaffold and was transferred back into the skin chamber to seed human keratinocytes on top. For this step, 500,000 keratinocytes/cm² of dermal surface area was introduced into the skin chamber followed by continuous rotation of the skin chamber for 4 hours at 37° C. In this example, the rotation step provides uniform seeding of keratinocytes on the surface of the dermis.

After cell attachment, the whole tissue with the skin scaffold was removed from the skin chamber, and submerged in epidermis culture medium for the proliferation of keratinocytes on the surface for 7 days. To bring the whole tissue into air-liquid interface, the ports were connected to plastic tubing and the tissue was suspended in a glass bottle. The tubes were connected to a medium reservoir and the skin scaffold was perfused with cornification medium using a peristaltic pump. After 10 days in the air liquid interface, wearable skin sleeves with proper dermis and epidermis formed. To explant the skin from the skin scaffolds as an intact piece, a horizontal surgical incision was made on the larger circular ends of the hindlimb. The WEHS was then taken off by simply pulling from the opposite ends with blunt forceps.

### Example 3 - Human Skin-Gloves

Using the methods described herein, an exemplary skin scaffold in the shape of a generic human-scale hand was designed using CAD software (e.g., SolidWorks and nTopology) and 3D-printed using a biocompatible polycarbonate-like material (KeySplint Soft; keyprint). The skin dermis was made using a PDMS skin chamber in the shape of the skin scaffold. Human keratinocytes were seeded on top as described above in rotation culture and the tissue was brought into the air-liquid-interface as described above for the formation of the epidermis.

### Example 4 - Air-Liquid Interface Culture for Other Organs

The exemplary method described here allows for creating an air-liquid-interface culture and therefore can directly be used or easily adapted for engineering other epithelial tissues that require air-liquid interface culture for their proper generation. These tissues may include the lungs, airways, and alveoli or the oral, nasal and middle ear epithelium.

To generate the oral, nasal, and middle ear epithelium, the methods described herein can be implemented using the epithelial cells of the oral mucosa, nasal and middle ear epithelium, respectively, similar to using keratinocytes for the skin. To generate the lungs, airways, or alveoli, the methods described herein can be adapted to include the airway or lung epithelium and their respective culture medium. To generate the airways and alveoli, the shape of the scaffold can be made cylindrical or spherical, respectively, to mimic the physiological shapes of these tissues. In addition, the methods described here for the dermis and the epidermis can later be adapted to also include the other underlying tissues, such as the hypodermis, skeletal muscles, cartilage and bones.

### Example 5 - Forming Wearable Engineered Human Skin (WEHS)

Another exemplary method of forming WEHS is described below.

Step 1: Acquire 3D computer aided drawing (CAD) model of the target area.

A patient-specific model of the target area or target location (e.g., hand) can be created by scanning the target area using a commercial 3D scanner (e.g., Creality CR-Scan). Alternatively, a generic model can be acquired through online CAD repositories (grabcad.com) or third-party sources (e.g., Zygote) for specific body parts of interest.

Step 2: Design and 3D-print a hollow and perfusable “skin scaffold”.

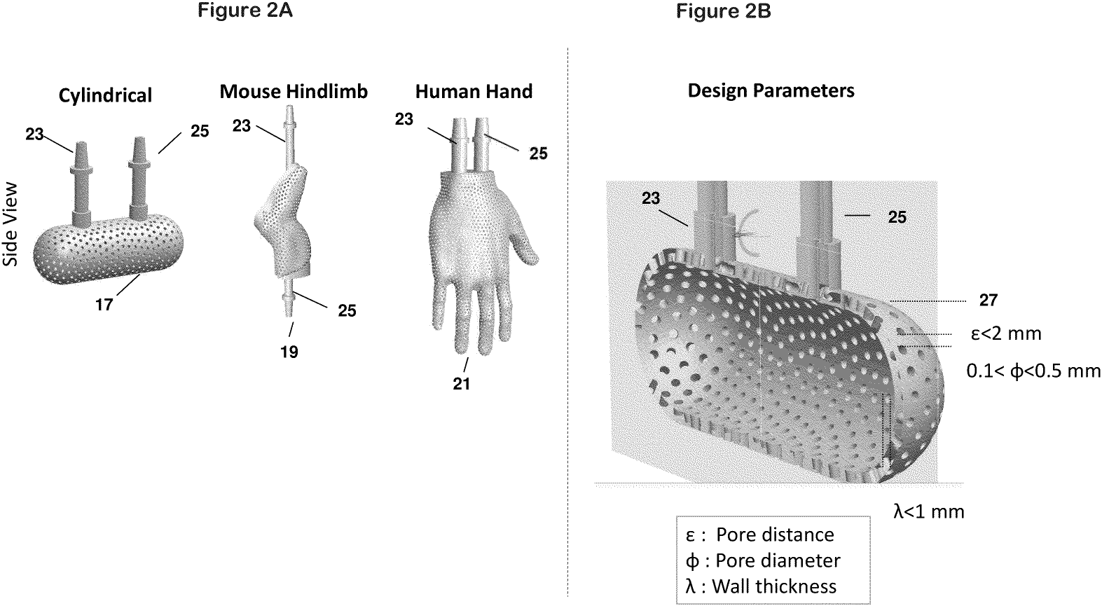

A skin scaffold was designed following the geometrical features of an acquired CAD model of a body part. FIG. 2A shows exemplary shapes for a skin scaffold including, but not limited to, cylindrical skin scaffold 17, mouse hindlimb skin scaffold 19 and human hand scaffold 21. It is understood that the shape of the skin scaffold can be determined by the need of a particular subject. For example, the acquired CAD model can be shaped with substantially the same geometry of a target area on the subject in need of treatment.

These exemplary skin scaffolds are hollow, porous, and perfusable in order to form the skin substitute in an air-liquid interface exposed to culture medium on the dermal side below and to air from the epidermal side above. Forming the skin in an air-liquid interface (e.g., with one side exposed to air and one side exposed to liquid) can promote proper formation of the epidermis layer. FIG. 2A shows a cylindrical shape 17 skin scaffold having an inlet port 23 and an outlet port 25 to allow for perfusion of the skin scaffold with cell culture media. A mouse hindlimb skin scaffold 19 and a human hand skin scaffold 21 are also shown with inlet port 23 and outlet port 25.

FIG. 2B shows a cross section of cylindrical skin scaffold 17 having pores 27, inlet port 23 and outlet port 25. Exemplary pore dimensions are also provided including a pore distance of < 2 mm, a pore diameter of between 0.1 and 0.5 mm, and a wall thickness of less than 1 mm. The density, size and uniform distribution of the pores on the surface of the skin scaffold can be adjusted to permit a sufficient amount of diffusion of cell culture medium inside the scaffold, for example, to the epidermis exposed to the air.

A 3D-printer (e.g., Carbon Printer; Material:Keysplint Soft) was used to create skin scaffolds. In this example, the pore diameter, pore distance and wall thickness were 0.5 mm, 1.5 mm, and 0.7 mm, respectively, with even distribution of pores on the surface.

In some instances, in which the overall size of the skin scaffold is large (e.g., human hand), or the pore size of the scaffold has to be larger than the recommended 0.5 mm due to technical limitations (e.g., 3D-printing system available), the skin scaffold can be alternatively coated with 5% w/v gelatin in water by briefly dipping the scaffold in the gelatin solution at room temperature, incubating it at 4° C. overnight and crosslinking it with 1% transglutamase at 37° C. for 2-4 hours. This process provides additional protection against potential undesired leakage of culture medium through the pores at later steps in the protocol when perfusion starts.

Step 3: Making a Skin Chamber Based on the Skin Scaffold

Next, a skin chamber that is custom designed to fit the scaffold was formed. A skin chamber comprising a top part and a bottom part was assembled by inserting the top part into the bottom part. The skin chamber was 3D-printed with a thermoplastic material (e.g., poly lactic acid (PLLA)). It is understood that any suitable material can be used for making the skin chamber. In this example, the skin chamber has an inner housing with the same geometry (e.g., substantially similar dimensions of the outer surface) as the skin scaffold, with an offset of 4 mm evenly from all surfaces of the skin scaffold. The skin scaffold was attached and suspended in the center of the skin chamber through inserting the inlet and outlet ports of the skin scaffold into two openings on the skin chamber wall.

Step 4: Cast the Dermis in the Skin Chamber Around the Skin Scaffold.

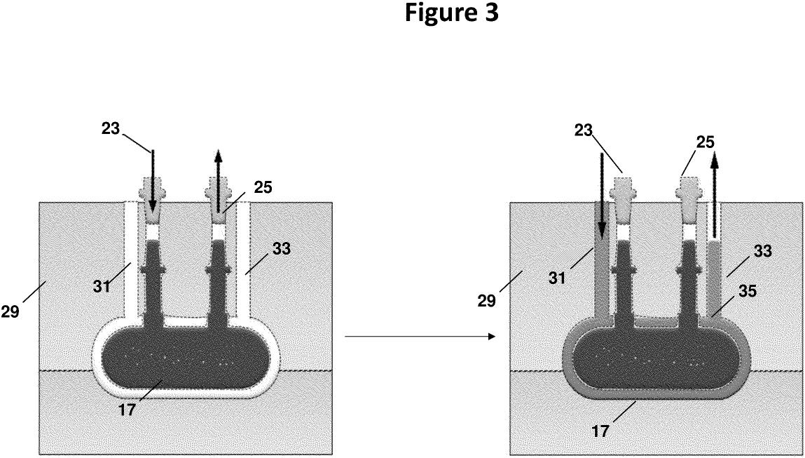

FIG. 3 shows cylindrical skin scaffold 17 suspended in skin chamber 29. Skin chamber 29 has skin chamber inlet port 31 and skin chamber outlet port 33. As shown in FIG. 3, the dermis solution composed of neutralized collagen type I gel (3 mg/ml) and dermal fibroblasts at a final cell density of 250,000 cells/ml was introduced into the skin chamber by pipetting the solution into skin chamber inlet port 31 and incubated for 1 hour at 37° C. to form gel 35 around cylindrical skin scaffold 17.

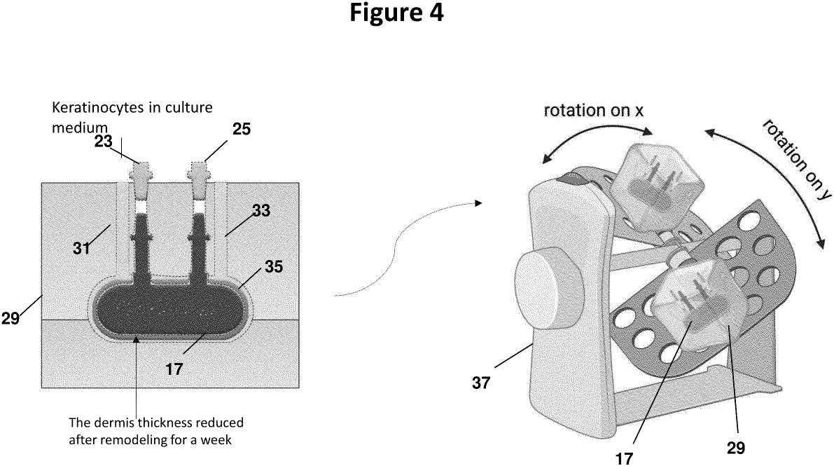

The skin scaffold with the formed dermis was taken off the skin chamber and submerged in a fibroblast culture medium for 2 weeks to promote formation and remodeling of the dermis. As indicated in FIG. 4, left panel, during the incubation period in the fibroblast culture medium, the dermis contracted to 25 to 33% of its initial thickness.

Step 5: Seed the Epidermal Cells on the Dermis in the Skin Chamber.

After 2 weeks, the skin scaffold with the formed dermis was placed back into the skin chamber to seed human neonatal keratinocytes on top. In this step, 3-5 million keratinocytes in keratinocyte culture medium were introduced into the skin chamber by pipetting into skin chamber inlet port 31 (FIG. 4, left panel). Subsequently, the skin chamber was rotated continuously on rotating platform 37 for 4 hours at 37° C. on x and y axes (2 hours each axis) at a speed of 5 rotations per minute for uniform seeding of keratinocytes on the surface of the dermis (FIG. 4, right panel). The skin chamber can be rotated discontinuously with breaks between period of continuous rotation. In some instances, the chamber can be rotated at any suitable speed (e.g., 1-10 rotations/minute, 5 rotations/min).

After keratinocyte attachment, the assembly of skin scaffold, dermis, and keratinocytes was removed from the skin chamber and submerged in epidermis culture medium for the proliferation of keratinocytes on the surface for up to 7 days.

Step 6: Perfuse to Achieve Air-Liquid-Interface Culture for Epidermalization.

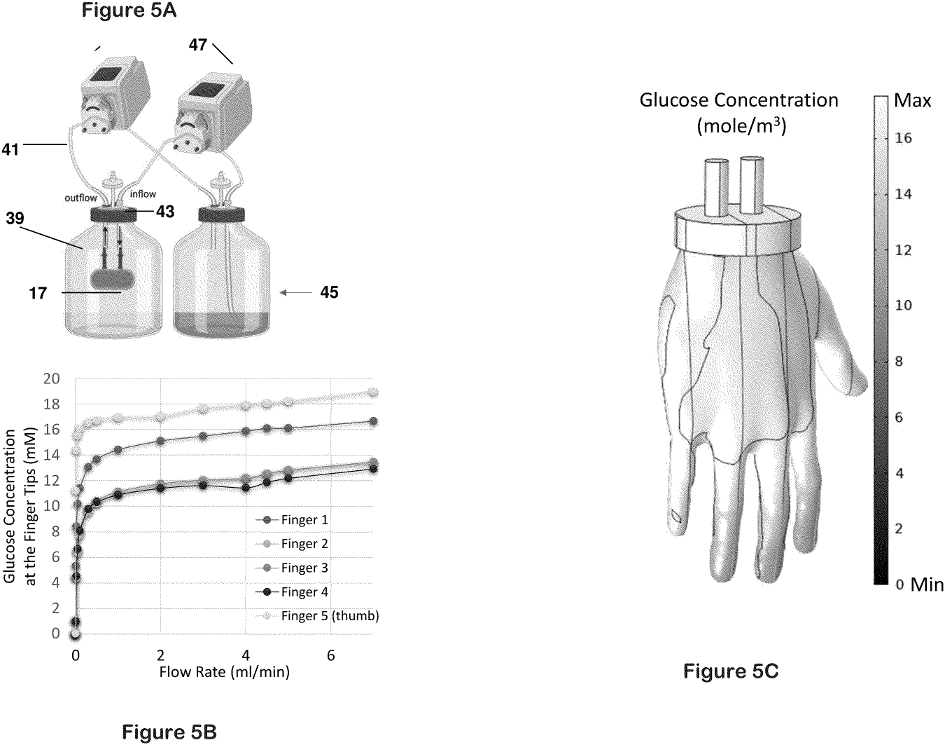

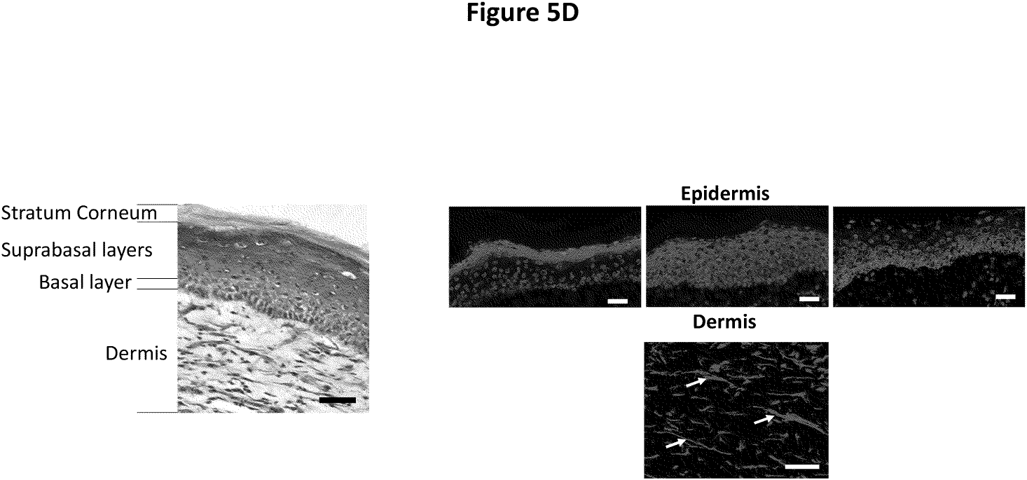

The system shown in FIG. 5A can be used to create an air-liquid interface for the proper formation of dermis. As shown in FIG. 5A, the assembly of cylindrical skin scaffold 17, dermis, and keratinocytes was transferred and suspended in the air in glass bottle 39 by connecting inlet port 23 and outlet port 25 to plastic tubing 41 attached to the bottle cap 43. Medium reservoir 45 can provide cell growth media to the cylindrical skin scaffold 17. Plastic tubing 41 is shown connected to medium reservoir 45, and cylindrical skin scaffold 17 was perfused with cornification medium using peristaltic pump 47 at a predetermined optimal flow rate.

The optimal flow rate was computationally estimated according to each skin geometry through simulations of molecular transport using COMSOL Multiphysics Software based on the distribution of the glucose to the extremities of the skin geometry (e.g., fingertips of the hand (FIGS. 5B-5C). FIG. 5B estimates the flow rate of the distribution of glucose to each finger of an exemplary hand skin scaffold. FIG. 5C is a graphical representation of the estimated flow rate to each finger. In this example, optimizing the flow rate for the medium to perfuse the dermis inside and providing air exposure outside can promote proper differentiation of keratinocytes and formation of the epidermis.

After 10 days in air-liquid-interface culture, wearable engineered human skin (WEHS) with proper dermis and epidermis were formed. FIG. 5D is an H&E stain (hematoxylin and eosin) and immunofluorescence (epidermis red) showing the presence of the dermis (lower portion) and epidermis layers (top portion) indicating that proper skin layers were formed using the methods described herein. The proper formation of the skin was assessed by analyzing the spread and elongated morphology of the dermal fibroblasts in the dermis (shown by the pattern of F-actin staining as indicated by the white arrows in FIG. 5D) and by the presence of the specific layers of the epidermis, e.g., basal layer (innermost layer), suprabasal layer and stratum corneum (outermost layer), based on their morphology in the H&E staining (the basal layer: vertically aligned first line of epidermal cells; suprabasal layers: horizontally oriented cells above the basal layer; stratum corneum: cornified top layers) and expression of layer-specific markers (K14: basal layer; K10: suprabasal layers; Loricrin: stratum corneum).

Step 7: Seed Vascular Cells by Perfusion for Vascularization.

An exemplary vascularization of WEHS was performed to promote the integration and viability of the grafts. Endothelial cells were injected into the circulating culture medium at a cell density of 5 million/ml so that they can enter the skin scaffold through perfusion. Endothelial cells then attached on the inner walls of the dermis through the pores after 3 hours of static culture at 37 C° and were stimulated by growth factors in the medium to form spontaneous vessel-like structures sprouting from the pores for 2-3 days prior to grafting.

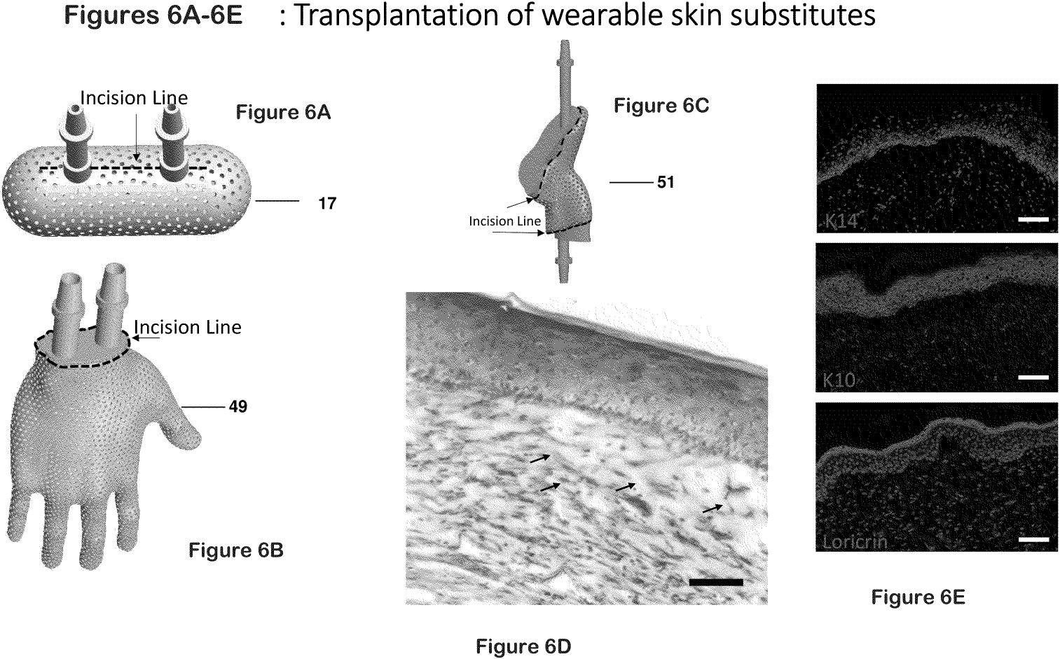

To explant the WEHS from the skin scaffold as a single piece, a minimal surgical incision was made on the WEHS following the incision line determined for each skin geometry. WEHS were peeled off of the skin scaffold as an intact shape. The incision site depends on the shape of the skin. For example, for skin transplantation on the hands, a circular incision would be made only around the elbow area to take off the WEHS to be grafted onto patients. FIGS. 6A-6C show exemplary incision lines for a cylindrical skin scaffold 17, a human hand skin scaffold 49, and a mouse hindlimb 51.

To use WEHS for in vitro drug testing purposes instead of grafting, WEHS can be kept on skin scaffolds under perfusion. Chemicals or drugs can be directly added to the medium or on the epidermis to mimic systemic or topical treatment, respectively.

### Example 6 - Transplantation of Wearable Skin Substitutes

To graft the WEHS, incisions can be made on the skin scaffold as shown, for example, in FIG. 6A (cylindrical skin scaffold 17), FIG. 6B (human hand skin scaffold 49), and FIG. 6C (Mouse Hind Limb 51). Exemplary incision lines are shown to permit removal of the WEHS prior to grafting on to the target location. It is understood that a skin scaffold can be configured to adapt to the shape of any target location as described herein and incision sites can be determined by a doctor or medical professional for grafting on to the target location.

For example, a cylindrical piece of mouse skin with a height and diameter of 1 cm and 0.6 mm, respectively, was removed from the upper hindlimb area with a single vertical (1 cm) and two horizontal incisions (0.3 mm each). The WEHS was put on the recipient by inserting the paw and hindlimb through the holes on each end of the WEHS. 3 sutures (size 5-0) on both ends was used to secure the skin in place. The tissue was harvested after 2 weeks and the formation of the skin was examined by hematoxylin and eosin staining (FIG. 6D) and immunofluorescent staining of keratin 14 (for the basal layer), K10 (for the suprabasal layer) and loricrin (for the cornified layer) (FIG. 6E from top to bottom panel).

The proper formation of the skin was assessed by the spread and elongated morphology of the dermal fibroblasts in the dermis (shown by the pattern of H&E staining indicated by the black arrows in FIG. 6D) and by the presence of the specific layers of the epidermis (e.g., basal layer (innermost layer)), suprabasal layer and stratum corneum (outermost layer). In addition, proper formation of skin was based on expression of layer-specific markers (K14: basal layer; K10: suprabasal layers; Loricrin: stratum corneum) as shown in FIG. 6E.

### Example 7 - Comparison of the WEHS to Conventional Engineered Skin

The methods and resulting engineered skin substitutes (WEHS) described herein significantly enhance dermal extracellular matrix and epidermal basement membrane remodeling compared to the conventional methods. Thus, not only are the WEHS custom shaped for grafting on to a specific target location, but they are also biologically more similar to actual skin, and their use is more likely to be clinically successful.

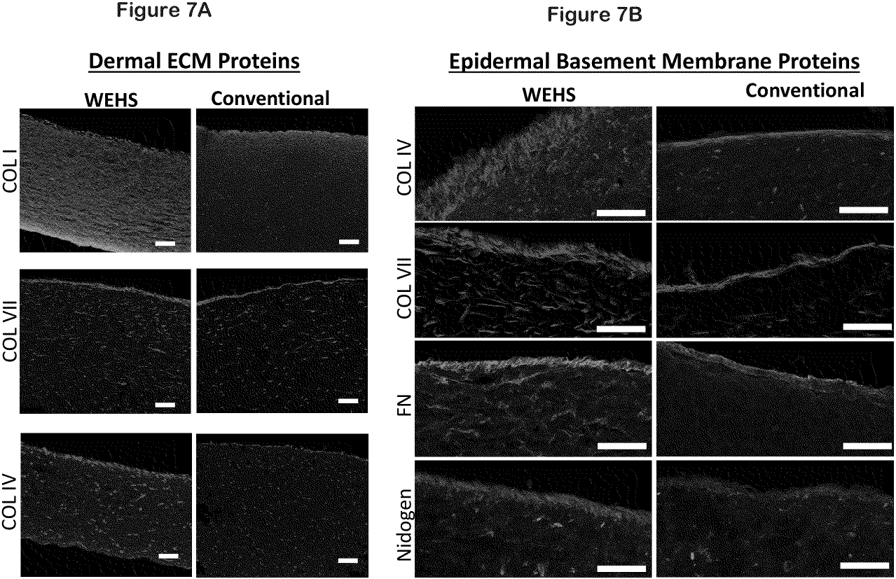

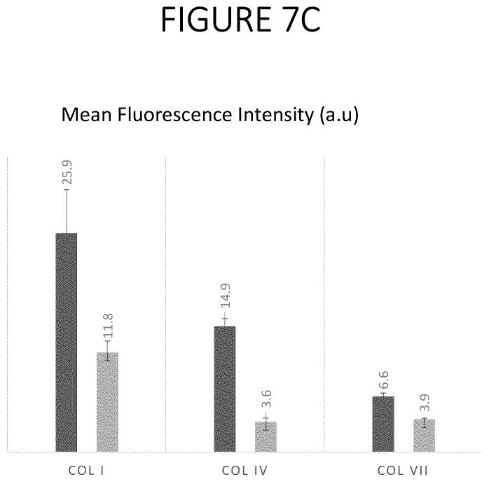

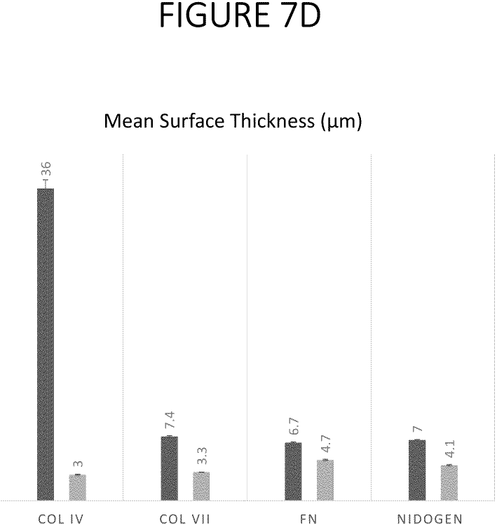

FIG. 7A shows exemplary immunofluorescent staining images of the histological sections of the dermal compartments of WEHS and conventional engineered skin stained for Collagen I, VII and IV, all of which are major components of the human dermis. WEHS exhibit a significantly higher production and deposition of these proteins in the dermal compartment of the constructs compared to skin substitutes made according to conventional methods. Conventional skin substitutes were made in accordance with FIG. 1 and as described, for example, in P. Gangatirkar, S. Paquet-Fifield, A. Li, R. Rossi, P. Kaur, Nat. Protoc. 2007, 2, 178. See, also, U.S. Pats. 6,497,875; 4,485,096; 6,039,760, and CN100522264C. Higher production and deposition of collagen I, VII, and IV as well as more lateral organization of collagen fibers (as opposed to more orthogonal in conventional) in the dermis shows the skin substitutes made according to the methods described herein are closer to actual human skin and are more likely to be accepted after transplantation and maintain normal function compared to conventional skin substitutes. Since the fibers of the WEHS are aligned in the lateral direction, they can oppose to the applied stretching force and withstand higher mechanical stress. As a result, during grafting surgery, the surgeon can more easily handle the graft, and suture it without rupturing.

FIG. 7B provides exemplary immunofluorescent high magnification images of the important epidermal basement membrane proteins, e.g., COLIV, COLVII, Fibronectin (FN) and Nidogen. The level and localization of all these proteins were more pronounced in WEHS compared to the proteins in conventional skin substitutes. The WEHS generated an increased localization of dermal fibroblasts on the top surface of the dermis (2-5 layers in WEHS vs. 1 -2 layers in conventional) and a thicker and denser layer of basement membrane ECM proteins critical for epidermis attachment, formation and homeostasis, compared to conventional skin substitutes. In addition, the WEHS generated a mesh-like ECM protein organization on the dermal surface, an important physiological characteristic of the basement membrane proteins in human skin for the firm attachment and function of the epidermis and a feature that is not represented in the conventional model. Collectively, this data shows the skin substitutes made according to the methods described herein are closer to actual human skin and are more likely to be accepted after transplantation and maintain normal function compared to conventional skin substitutes.

FIG. 7C provides the mean fluorescence intensity of collagen I, VII, and IV stained for in FIG. 7A and compares the results for WEHS (dark gray bars) and conventional skin substitutes (light gray bars). The data show that the fluorescence intensity for these proteins in WEHS is higher than conventional skin substitutes.

FIG. 7D provides the mean thickness of the layer covered by the fluorescently-labelled proteins in FIG. 7B (COLIV, COLVII, Fibronectin (FN) and Nidogen) and compares the results for WEHS (dark gray bars) and conventional skin substitutes (light gray bars). The data show that the thickness of the layer covered by these proteins in WEHS is higher than conventional skin substitutes.

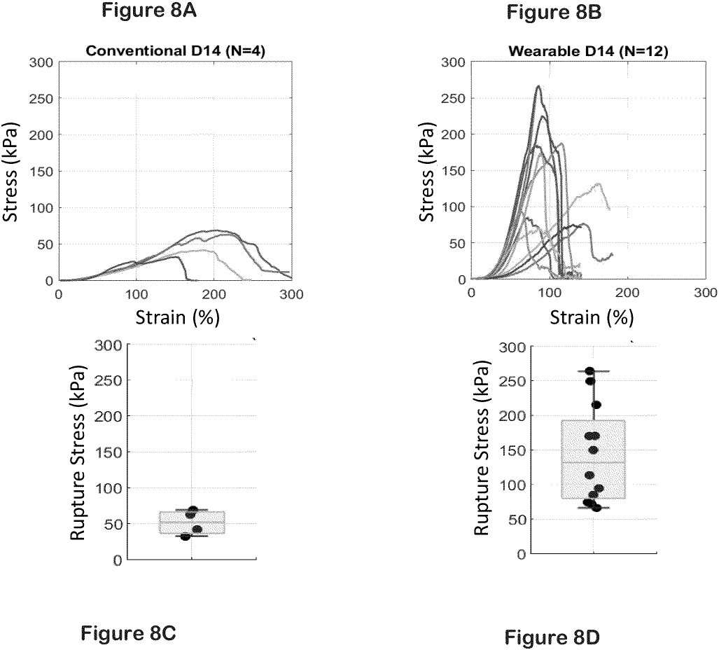

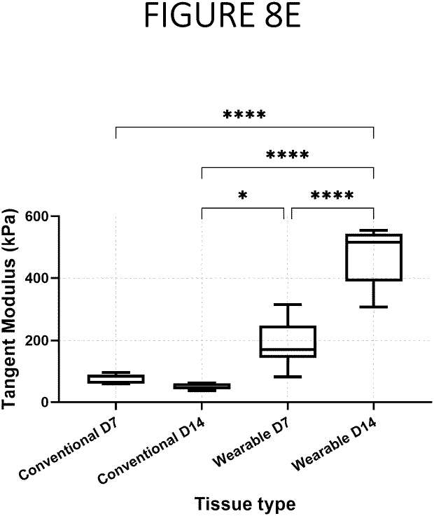

FIGS. 8A-8E shows that skin substitutes as described herein (e.g., WEHS) have significantly enhanced dermis mechanical properties compared to the skin substitutes made according to conventional methods. The dermis of the wearable and conventional constructs was mechanically stretched vertically and the mechanical properties such as stress, strain and rupture stress, Young’s modulus were measured and calculated. Wearable constructs were made as described herein. Conventional constructs were made following the method described in FIG. 1 by using collagen type I as the 3D hydrogel and cells as dermal fibroblasts and keratinocytes, same material and cell types and same batches and cell sources used in parallel to make WEHS. As shown in FIGS. 8, WEHS dermis can withstand significantly higher levels of rupture stress compared to conventional dermis.

Four conventional skin substitutes were subjected to mechanical stress (FIG. 8A) and withstood up to 60 kPa of mechanical stress. In contrast, twelve WEHS made in accordance with the methods described herein withstood up to 260 kPa of mechanical stress as shown in FIG. 8B. Four conventional skin substitutes were subjected to rupture stress (FIG. 8C) and withstood up to 60 kPa of rupture stress. In contrast, twelve WEHS made in accordance with the methods described herein withstood up to 260 kPa of rupture stress as shown in FIG. 8D - about a four-fold difference. The average rupture stress tolerance of WEHS was 135 kPa compared to an average rupture stress tolerance of 52 kPa for conventional skin substitutes.

The higher level of lateral organization of dermal ECM fibers in WEHS, as shown in FIG. 7A, may have contributed to the enhanced mechanical properties observed here for WEHS. The WEHS made in accordance with aspects described herein can withstand significantly more mechanical and rupture stress than conventional skin substitutes, and allow for better handling and suturing during transplantation and a lower risk of graft rupturing following the surgery.

FIG. 8E shows exemplary Young’s modulus indicating the contribution of fibrous ECM to the overall mechanical strength of the material. FIG. 8E shows significantly increased tangent modulus (kPA) in WEHS (wearable) compared to conventional skin substitutes. “D7” and D14” refer to the number of days the dermal part of the skin was left in submerged culture for remodeling. When fibroblasts are encapsulated in collagen, they are only surrounded by collagen type I at first. As time progresses, the fibroblasts continue to remodel the ECM, and express other proteins as shown, for example, in FIG. 7A. Permitting additional remodeling from day 7 to day 14 increased the mechanical strength of the WEHS as shown in FIG. 8E (compare Wearable D7 to Wearable D14)

While the aspects described herein have been disclosed with reference to certain embodiments, numerous modifications, alterations, and changes to the described aspects are possible without departing from the sphere and scope of the present invention, as defined in the appended claims. Accordingly, it is intended that the present invention not be limited to the described aspects, but that it has the full scope defined by the language of the following claims, and equivalents thereof.

What is claimed is:

1. A skin substitute comprising an outer-facing portion and an inner-facing portion, wherein the skin substitute is configured to conform to a shape and a dimension of a body part of a subject, and wherein the skin substitute has at least one surface that circles back on itself so as to enclose at least a portion of the body part.

2. The skin substitute of claim 0, wherein the outer-facing portion is an epidermal portion, and the inner-facing portion is a dermal portion.

3. The skin substitute of claim 0, wherein the epidermal portion comprises epidermal cells.

4. The skin substitute of claim 0, wherein the epidermal cells are selected from the group consisting of keratinocytes, melanocytes, Langerhan cells, and epidermal stem cells.

5. The skin substitute of claim 0, wherein the dermal portion comprises dermal cells.

6. The skin substitute of claim 0, wherein the dermal cells are selected from one or more of fibroblasts, mesenchymal cells, dermal papilla cells, adipocytes, sensory neurons, mesenchymal stem cells, endothelial cells, smooth muscle cells, and pericytes.

7. A method of making a skin substitute, comprising forming the skin substitute on or in a hollow and porous scaffold, wherein the skin substitute has an outer-facing portion and an inner-facing portion, wherein the skin substitute is configured to conform to a shape and a dimension of a body part of a subject, and wherein the skin substitute has at least one surface that circles back on itself so as to enclose at least a portion of the body part.

8. The method of claim 7, wherein the scaffold further comprises an inlet port and an outlet port arranged so that a liquid can be introduced into and removed from an interior of the scaffold, wherein the liquid forms an air/liquid interface at one or more walls of the scaffold.

9. The method of claim 8, wherein the liquid comprises one or more of dermis culture medium, epidermis culture medium, cornification medium, endothelial cell culture medium, and skin and vasculature co-culture medium.

10. The method of claim 8, further comprising forming a chamber for receiving the scaffold, and placing the scaffold into the chamber.

11. The method of claim 10, further comprising introducing epidermal cells into the chamber, wherein an epidermal monolayer is formed on the scaffold in epidermis culture medium.

12. The method of claim 11, further comprising introducing laminin and fibronectin into the chamber.

13. The method of claim 11, wherein the chamber is rotated after introducing the epidermal cells into the scaffold.

14. A method of forming a wearable skin substitute, the method comprising:
obtaining a three-dimensional model of a target region of a subject’s body;
forming, based on the three-dimensional model, a hollow, porous, and perfusable scaffold that conforms to the target region, the scaffold having an outer surface and a plurality of pores;
forming, based on the three-dimensional model, a chamber having an inner surface dimensioned to enclose the outer surface of the scaffold, with a spacing of 2-7 mm between the outer surface of the scaffold and the inner surface of the chamber;
positioning the scaffold inside the chamber;
forming a dermis in the chamber by introducing a dermis solution comprising a collagen gel and dermal fibroblasts into the chamber, wherein the dermis is formed around the scaffold;
seeding epidermal cells on the dermis in the chamber; and
perfusing the scaffold with medium to form an air-liquid interface culture.

15. The method of claim 14, wherein a density, a size, and a distribution of the pores on the surface of the scaffold are configured to permit diffusion of cell culture medium inside the scaffold to the dermis exposed to air.

16. The method of claim 14, further comprising perfusing the dermis with endothelial medium comprising endothelial cells and a growth factor.

17. The method of claim 14, wherein the inner surface of the chamber is dimensioned so that the spacing between the outer surface of the scaffold and the inner surface of the chamber is 3 to 5 mm and is substantially uniform.

18. The method of claim 14, wherein the scaffold comprises an inlet port and an outlet port for perfusing the scaffold with medium.

19. The method of claim 14, wherein the dermis solution is introduced into the chamber, the scaffold is incubated in the dermis solution, and the dermis solution forms a gel around the scaffold.

20. The method of claim 14, wherein the chamber is rotated continuously for at least 4 hours.
